(19) United States
(12) Patent Application Publication
(10) Pub. No.: US 2011/0318270 A1
(43) Pub. Date: Dec. 29, 2011

(54) DRUG IDENTIFICATION PROTOCOL FOR TYPE 2 DIABETES BASED ON GENE EXPRESSION SIGNATURES

(75) Inventors: Ken Walder, Victoria (AU); Nicky Konstantopoulos, Victoria (AU); Greg Royce Collier, Victoria (AU)

(73) Assignee: VERVA PHARMACEUTICALS LTD, SOUTHBANK, VICTORIA (AU)

(21) Appl. No.: 13/203,826

(22) PCT Filed: Feb. 25, 2010
(86) PCT No.: PCT/AU2010/000221

§ 371 Date: Aug. 29, 2011

Related U.S. Application Data
(60) Provisional application No. 61/156,149, filed on Feb. 27, 2009.

(51) Int. Cl. A61K 49/00; A61K 31/7088; A61P 3/10; G01N 33/566; A61K 38/02; C12N 15/11; C12Q 1/68

(52) U.S. Cl. 424/9.1; 536/23.1; 514/44 R; 435/6.12; 435/7.92; 514/6.9

(57) ABSTRACT

It relates generally to the field of drug identification and evaluation and therapeutic optimization. More particularly, it provides a protocol for identifying compounds useful in the treatment of TNFα associated diabetes or a condition associated with diabetes based on a signature of genomic or proteomic expression. Diagnostic and prognostic protocols for diabetes and conditions associated therewith are also provided. Further, optimization of therapeutic intervention is also provided.

### FIELD

The present invention relates generally to the field of drug identification and evaluation and therapeutic optimization. More particularly, the present invention provides a protocol for identifying compounds useful in the treatment of TNFα associated diabetes or a condition associated with diabetes based on a signature of genomic or proteomic expression. Diagnostic and prognostic protocols for diabetes and conditions associated therewith also form part of the present invention. Optimization of therapeutic intervention is also encompassed by the present invention.

### BACKGROUND

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in any country.

A Gene Expression Signature (GES) and corresponding Proteomic Expression Signature (PES) provide information on clusters of co-ordinately expressed genes (Alizadeh et al. Nature 403:503-511, 2000) and can be used to describe different biological or physiological states (van de Vijver et al. N Engl J Med 347.1999-2009, 2002). GES's have been used in cancer biology to assist in tumor classification, prognosis prediction and patient response to therapeutic intervention (Cooper et al. Nat Clin Pract Urol 4:677-687, 2007; Nuyten and van de Vijver Semin Radiat Oncol 18:105-114, 2008). Whilst single gene expression-based screening approaches have been used to identify drugs that regulate metabolic gene targets such as PGC-1α (Arany et al. Proc Natl Acad Sci USA 105:4721-4726, 2008), a GES represents a group of genes whose mRNA expressions are instructive of the integrated response of a cell to its environment. Hence, a GES is obtained irrespective of any genes in the cluster. Therefore, these genes may not directly regulate the changed metabolic state; rather, they may be representative markers of it. Hence, instructive assays can be designed without the need to ascribe gene function.

Type 2 diabetes (T2D) is epidemic and is a major health issue world-wide. A key feature of this disease is insulin resistance. The causes of insulin resistance appear multifactorial with high levels of circulating non-esterified fatty acids, chronic inflammation, and endoplasmic reticulum and oxidative stress all potentially contributing (Mlinar, et al. Clin Chim Acta 375:20-35, 2007). The pro-inflammatory cytokine tumor necrosis factor-alpha (TNFα) is implicated in the induction of insulin resistance seen in obesity and T2D as elevated TNFα levels function both in an autocrine and paracrine fashion to reduce insulin sensitivity in several tissues including adipose tissue (Hotamisligil, Nature 444:860-867, 2006; Ruan and Lodish Cytokine Growth Factor Rev 14:447-455, 2003). TNFα secreted by adipocytes and can decrease their insulin sensitivity by various mechanisms including induction of lipolysis and fatty acid release, impairing insulin signalling and reducing GLUT4 levels (Ruan and Lodish 2003 supra). The actions of TNFα are mediated by several kinases including p38 MAP and Jun-N-terminal kinases, protein kinase C (PKC), nuclear factor kappa B (NFKB) activation and the down-regulation of peroxisome proliferator-activated receptor gamma (PPATγ) [Qi and Pekala Proc Soc Exp Biol Med 223:128-135, 2000; Tang et al. Proc Natl Acad Sci USA 103:2087-2092, 2006]. Agents such as aspirin (ASA) and the thiazilodinedione troglitazone (TGZ) can improve insulin sensitivity in vivo (Miles et al. Diabetes 46:1678-1683, 1997; Yuan et al. Science 293:1673-1677, 2001), and in vitro, they appear to counteract the impact of TNFα via multiple pathways (Gao et al. J Biol Chem 278:24944-24950, 2003; Ohsumi et al. Endocrinology 135:2279-2282, 1994; Peraldi and Spiegelman J Clin Invest 100:1863-1869, 1997). Therefore, agents which reverse the effects of TNFα in adipocytes have the potential to improve whole-body insulin sensitivity.

Due to the ever increasing incidence of diabetes in society, there is an urgent need to identify drugs useful in treating or ameliorating the symptoms of diabetes as well as diagnosing and monitoring diabetes or a condition associated therewith, such as obesity, blindness, nephropathy and/or cardiovascular disease.

### SUMMARY

In accordance with the present invention, a genomic/proteomic approach is applied to define a biological or physiological state associated with diabetes such as T2D and in particular TNFα associated insulin resistant T2D. Specifically, a GES is established which reflects the TNFα associated insulin resistance or sensitivity state of a cell and this is used to screen for insulin sensitizing agents and to identify or monitor TNFα associated T2D in a subject. In one embodiment, a GES is generated in cells rendered insulin resistant by TNFα and then “insulin re-sensitized” by post-treatment with ASA and TGZ. This model is consistent with the human condition where individuals are typically treated with specific drugs following diagnosis of the disease. The use of both ASA and TGZ ensures the activation of multiple signalling pathways in the reversal of insulin resistance. Using gene expression profiling, the GES identified, whose expression is statistically different in the insulin resistant versus the insulin re-sensitized state comprises two or more of the genetic biomarkers PKM2, Skp1a, CD63, STEAP4, ACS1, CS and/or CLU. The mRNA expression of these genes is used as the basis to screen a drug library to search for potential insulin sensitizing compounds. Compounds identified by the GES and their drug classes are validated both in vitro and in vivo to determine their insulin sensitizing capabilities. Reference to a “GES” includes determining gene expression levels via its corresponding proteomic expression signature or PES. Protein detection assays may be used to determine a PES.

Hence, the present invention provides a panel of 2 or more and in particular from 2 to 7 genetic biomarkers which are useful in the generation of a GES (or corresponding PES) which is associated with a biological or physiological state of insulin sensitivity or resistance in T2D, and in particular TNFα associated T2D. The GES is also predictive of a predisposition to develop T2D or the probability of a subject developing a condition associated with T2D, such as obesity, blindness, nephropathy and/or cardiovascular disease and in particular TNFα associated T2D. In one aspect, the panel of 2 or more biomarkers of the present invention is differentially expressed such that in subjects with TNFα associated T2D or who are developing TNFα associated insulin resistance, gene expression levels of PKM2, Skp1a, CD63, STEAP4 and CLU are increased whereas ACS1 and CS are decreased. Drugs are identified which induce a GES (or corresponding PES) characteristic of insulin sensitivity.

Accordingly, the present invention provides a gene expression signature (GES) or corresponding proteomic expression signature (PES) indicative of Type 2 diabetes or symptoms thereof, said GES or PES comprising expression levels of at least two genes or gene products selected from the list comprising PKM2, Skp1a, CD63, STEAP4, ACS1 (FACL2), CS and CLU.

Diagnosis and prognosis of TNFα associated T2D, a pre-disposition for TNFα associated T2D or a probability of developing a condition associated with TNFα associated T2D also form part of the present invention by determining the GES (or corresponding PES) based on the panel of from 2 to 7 of the genes. The ability to diagnose or prognose TNFα associated T2D, a pre-disposition for TNFα associated T2D or a probability of developing a condition associated with TNFα associated T2D has important implications for the treatment and/or management of a subject's condition such as in the monitoring of a therapeutic regime.

The genes or corresponding proteins in the GES are referred to herein as biomarkers. The present invention relates to the collective information obtained by the expression of 2 or more genes in the GES rather than relying on the expression of a single gene.

Reference to a “biomarker” includes a marker of TNFα associated T2D, a pre-disposition for diabetes or a probability of developing a condition associated with TNFα associated T2D, or a predisposition for developing TNFα associated T2D. The GES is formed by determining expression levels of a panel of 2 to 7 genes or their expression products. When screening proteinaceous products of the genes, a proteomic expression signature or PES is identified. Hence, the present invention encompasses a GES or PES of insulin resistance or sensitivity based on 2 or more of PKM2, Skp1a, CD63, STEAP4, ACS1, CS and/or CLU. Reference to “2 or more” or 2 to 7″ includes 2, 3, 4, 5, 6 or 7 of the above mentioned genes.

Reference to “TNFα associated T2D” or “TNFα associated insulin resistance or sensitivity” or “TNFα associated insulin resistant T2D” encompasses the spectrum of T2D conditions.

The present invention further enables optimization of therapeutic intervention for T2D by first stratifying a subject into a particular group based on a GES or corresponding PES and then selecting and administering a medicament having the same or similar GES/PES. The GES/PES may also be monitored over time and the medicaments changed based on maintaining a similar correlation between the subjects GES/PES and the selected medicament's GES/PES.

Accordingly, the present invention contemplates a method for stratifying a subject in need of treatment for Type 2 diabetes to facilitate therapeutic intervention, said method comprising determining a GES or corresponding PES for the subject comprising expression levels of at least two genes selected from PKM2, Skp1a1, CD63, ACS1 (FACL2), CS and CLU and selecting a medicament identified as a diabetes symptom reversing agent using the same or substantially similar GES or corresponding PES to the GES or PES used to stratify the subject.

The present invention further provides a method of treatment of a subject with Type 2 diabetes or symptoms thereof, said method comprising determining the GES or corresponding PES for the subject comprising expression levels of at least two genes selected from PKM2, Skp1a1, CD63, ACS1 (FACL2), CS and CLU and administering a medicament identified as a diabetes symptom reversing agent using the same or substantially similar GES or corresponding PES to the GES or PES determined on said subject.

Another aspect of the present invention relates to a method of treatment of a subject with Type 2 diabetes or symptoms thereof, said method comprising determining the GES or corresponding PES for the subject comprising expression levels of at least two genes selected from PKM2, Skp1a1, CD63, ACS1 (FACL2), CS and CLU and administering a medicament identified as a diabetes symptom reversing agent using the same or substantially similar GES or corresponding PES to the GES or PES determined on said subject and monitoring the GES or corresponding PES over time and adjusting the medication such that the medicament has a GES or corresponding PES the same or substantially similar to the last determined GES or PES for the subject.

The present invention contemplates the use of the GES or PES of TNFα associated insulin resistance or sensitivity in the manufacture of a medicament in the treatment of TNFα associated T2D or a condition associated therewith.

Accordingly, one aspect of the present invention provides a GES or corresponding PES, of a level of TNFα associated insulin resistance or sensitivity comprising genes selected from 2 or more of PKM2, Skp1a, CD63, STEAP4, ACS1 (also known as FACL2), CS and CLU or a homolog thereof wherein a state of insulin resistance is identified when expression in a cell of PKM2, Skp1a, CD63, STEAP4 and/or CLU is/are increased relative to a control and/or ACS1 and/or CS is/are decreased relative to a control.

A “control” in this context includes the expression levels in an insulin-sensitive cell.

In another embodiment, the present invention contemplates a GES or corresponding PES of a level of TNFα associated insulin resistance or sensitivity comprising genes selected from 2 or more PKM2, Skp1a, CD63, STEAP4, ACS1, CS and CLU or a homolog thereof wherein a state of TNFα associated insulin sensitivity is identified when expression in a cell of PKM2, Skp1a, CD63, STEAP4 and/or CLU is/are decreased relative to a control and/or ACS1 and/or CS is/are increased relative to a control.

In this aspect, the “control” includes the expression levels in an insulin-resistant cell.

The present invention may be conducted in situ or on a biological sample from the subject. Hence, the present invention further provides a method for the diagnosis or prognosis of TNFα associated T2D or a predisposition for the development of TNFα associated T2D or a complication associated with TNFα associated T2D in a subject, the method comprising: (a) obtaining a biological sample from a subject; (b) determining the GES or corresponding PES based on 2 or more of PKM2, Skp1a, CD63, STEAP4, ACS1, CS and/or CLU in the biological sample; and (c) comparing the GES in the biological sample to a statistically validated threshold, wherein the GES or its corresponding PES is instructive of the level of TNFα associated T2D insulin sensitivity or resistance.

Hence, the GES in one biological/physiological state of TNFα associated T2D insulin resistance or sensitivity is referred to herein as a knowledge base. By comparing the GES or corresponding PES between knowledge bases in the presence of agents or drugs, useful medicaments or the treatment of TNFα associated T2D are identified.

The present invention further contemplates, therefore, a method for identifying a compound which reduces the level of TNFα associated T2D insulin resistance in cells, the method comprising contacting TNFα associated T2D insulin resistant cells having a first GES or corresponding PES which is instructive of TNFα associated T2D insulin resistance (first knowledge base) and then screening for a second GES or corresponding PES which is instructive of TNFα associated T2D insulin sensitivity (second knowledge base) wherein a compound which promotes development of the second GES is selected as the compound.

The first and second knowledge bases may be determined in the assay or be part of a statistically validated control.

The present invention particularly relates to identifying TNFα associated T2D medicaments in the treatment of humans.

The use of a GES is more efficacious then the use of single gene indicators of T2D and this is particularly useful in monitoring therapy and screening for potential medicaments with insulin sensitizing properties.

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIG. 1 is a graphical representation of a summary of the small molecule library screen results using the TNFα-based GES. A. Ranking of average Zrcc score for each compound family with 10 or more members. The insulin re-sensitized TNFα plus TGZ and ASA (TTA) co-treated and insulin resistant TNFα-treated (TNF) controls are represented (*p<0.05 to TNF and ̂p<0.05 to TTA; n=10-62).

FIGS. 2a and 2b are graphical representations of a compound stimulation of HA-tagged GLUT4 translocation to the plasma membrane in 3T3-L1 adipocytes. Adipocytes were incubated with 10 μM of each compound for 20 h prior to acute stimulation with 0.5 nM of insulin and measurement of HA-tagged GLUT4 translocation to the plasma membrane. a. The effect of the compound classes closest to TNFα plus ASA and TGZ co-incubated samples GES profile (see FIG. 1) on HA-tagged GLUT4 movement. Data are presented as fold change compared with 0.5 nM insulin alone set at 1.0 and represent mean values±SEM; n=12-40 per class. b. Individual CAI members effect on GLUT4 movement. Each bar represents the mean values of duplicate samples+SD and is represented as fold change to 0.5 nM insulin value (set at ‘1’). *p<0.003 compared with 0.5 nM insulin alone. Negative and positive controls include 0 nM (p=1.51×10-13, n=32) and acute maximal 200 nM insulin (p=2.55×10-9, n=32), 20 h incubation of 10 μM TGZ (n=8) and 5 mM ASA (n=8) compared with 0.5 nM insulin alone, respectively.

FIGS. 3a to 3e are graphical representations of an effect of methazolamide on metabolic parameters in DIO and db/db mice. A. Change in blood glucose area under the curve (AUC) expressed as % to vehicle treated animals following an intraperitoneal glucose tolerance test in DIO mice treated with each corresponding drug at 50 mg/kg/d for 14 days. Abbreviations: 2-aminobenzene sulphonamide (2ABS), chlorthalidone (CTD), furosemide (FUR), dichlorphenamide (DCP), methazolamide (MTZ) and N-methyl-methazolamide (MMTZ). B. Dose-dependent effect of MTZ on blood glucose (top panel) and plasma insulin levels (lower panel) in DIO mice. Animals were treated with MTZ at the indicated doses for 14 days. *̂p<0.05 to vehicle (n=6) vs. 50 (n=5) and 100 (n=5) mg/kg, respectively. C. Dose-dependent effect of MTZ on fasting blood glucose levels in db/db mice. Mice were treated with vehicle (n=24) or 50 (n=23) mg/kg MTZ for 8 days. *p<0.05 to day 0 and ̂p<0.05 to corresponding vehicle. D. Dose-dependent effect of MTZ on glycosylated haemoglobin (Hb1Ac) in db/db mice following treatment with 50 mg/kg of MTZ for 28 days. Histograms represent the means±SEM, n=5-12. *p<0.05 to vehicle. E. Effect of MTZ and metformin combination on fasting blood glucose in db/db mice. Change in glucose levels in mice treated with vehicle, 20 mg/kg MTZ, 300 mg/kg metformin or 20 mg/kg MTZ and 300 mg/kg metformin for 8 days. *p<0.05 to vehicle, ̂p<0.05 to metformin alone.

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It must be noted that, as used in the subject specification, the singular forms “a”, “an” and “the” include plural aspects unless the context clearly dictates otherwise. Thus, for example, reference to “a GES” includes a single GES, as well as two or more GES's; reference to “an agent” includes a single agent, as well as two or more agents; reference to “the invention” includes a single or multiple aspects of an invention; and so forth.

The present invention identifies a cluster of genes, the collective expression of which, defines a GES (or corresponding PES) which is descriptive or instructive of a biological or physiological state associated with diabetes, and in particular TNFα associated T2D. More particularly, the biological or physiological state is the level of TNFα associated T2D insulin resistance or sensitivity of a cell. The GES or PES defining a particular state of TNFα associated T2D insulin resistance or sensitivity is referred to herein as a knowledge base. Hence, the progression from TNFα associated T2D insulin sensitivity to insulin resistance generates different knowledge bases. A comparison of these knowledge bases in the presence of agents enables the identification of agents which induce TNFα associated T2D insulin sensitivity in subjects.

The GES comprises expression information on 2 or more genes selected from PKM2, Skp1a, CD63, STEAP4, ACS1 (also known as FACL2), CS and CLU. Reference to “2 or more” or from “2 to 7” include 2, 3, 4, 5, 6 or 7 of these genes. Any and all combinations of 2 or more genes as listed above are encompassed by the present invention. In a particular embodiment, a first knowledge base is identified as TNFα associated T2D insulin resistance whereby expression of PKM2, Skp1a, CD63, STEAP4 and CLU is increased and expression of ASC1 and CS is decreased. A second knowledge base is identified for TNFα associated T2D insulin sensitivity whereby expression of PKM2, Skp1a, CD63, STEAP4 and CLU is decreased whereas expression of ACS1 and CS is increased.

Hence, the present invention also provides a GES or corresponding PES of a level of TNFα associated T2D insulin resistance or sensitivity comprising genes selected from 2 or more of PKM2, Skp1a, CD63, STEAP4, ACS1, CS and CLU or a homolog thereof wherein a state of TNFα associated T2D insulin resistance or sensitivity is identified when expression in a cell of PKM2, Skp1a, CD63, STEAP4 and/or CLU is/are increased relative to a control and/or ACS1 and/or CS is/are decreased relative to a control.

Accordingly, the present invention provides a gene expression signature (GES) or corresponding proteomic expression signature (PES) indicative of Type 2 diabetes or symptoms thereof, said GES or PES comprising expression levels of at least two genes or gene products selected from the list comprising PKM2, Skp1a, CD63, STEAP4, ACS1 (FACL2), CS and CLU.

A “control” in this context includes the expression levels in an insulin-sensitive cell.

In another embodiment, the present invention contemplates a GES or corresponding PES of a level of TNFα associated T2D insulin resistance or sensitivity comprising genes selected from 2 or more PKM2, Skp1a, CD63, STEAP4, ACS1, CS and CLU or a homolog thereof wherein a state of TNFα associated T2D insulin sensitivity is identified when expression in a cell of PKM2, Skp1a, CD63, STEAP4 and/or CLU is/are decreased relative to a control and/or ACS1 and/or CS is/are increased relative to a control.

In this aspect, the “control” includes the expression levels in an insulin-resistant cell.

The present invention further provides a method for the diagnosis or prognosis of TNFα associated T2D or a predisposition for the development of TNFα associated T2D or a complication associated with TNFα associated T2D in a subject, the method comprising: (a) obtaining a biological sample from a subject; (b) determining the GES or corresponding PES based on 2 or more of PKM2, Skp1a, CD63, STEAP4, ACS1, CS and/or CLU in the biological sample; and (c) comparing the GES in the biological sample to a statistically validated threshold, wherein the GES or its corresponding PES is instructive of the level of TNFα associated T2D.

The present invention further enables optimization of therapeutic intervention for T2D by first stratifying a subject into a particular group based on a GES or corresponding PES and then selecting and administering a medicament having the same or similar GES/PES. The GES/PES may also be monitored over time and the medicaments changed based on maintaining a similar correlation between the subjects GES/PES and the selected medicament's GES/PES.

Accordingly, the present invention contemplates a method for stratifying a subject in need of treatment for Type 2 diabetes to facilitate therapeutic intervention, said method comprising determining a GES or corresponding PES for the subject comprising expression levels of at least two genes selected from PKM2, Skp1a1, CD63, ACS1 (FACL2), CS and CLU and selecting a medicament identified as a diabetes symptom reversing agent using the same or substantially similar GES or corresponding PES to the GES or PES used to stratify the subject.

The present invention further provides a method of treatment of a subject with Type 2 diabetes or symptoms thereof, said method comprising determining the GES or corresponding PES for the subject comprising expression levels of at least two genes selected from PKM2, Skp1a1, CD63, ACS1 (FACL2), CS and CLU and administering a medicament identified as a diabetes symptom reversing agent using the same or substantially similar GES or corresponding PES to the GES or PES determined on said subject.

Another aspect of the present invention relates to a method of treatment of a subject with Type 2 diabetes or symptoms thereof, said method comprising determining the GES or corresponding PES for the subject comprising expression levels of at least two genes selected from PKM2, Skp1a1, CD63, ACS1 (FACL2), CS and CLU and administering a medicament identified as a diabetes symptom reversing agent using the same or substantially similar GES or corresponding PES to the GES or PES determined on said subject and monitoring the GES or corresponding PES over time and adjusting the medication such that the medicament has a GES or corresponding PES the same or substantially similar to the last determined GES or PES for the subject.

Reference to “a diabetes symptom reversing agent” includes an agent which reverses diabetes and in particular Type 2 diabetes.

A “biological sample” includes a biological fluid sample such as but not limited to whole blood, blood plasma, serum, mucus, urine, isolated peripheral blood mononuclear cells, lymphocytes, semen, faecal matter, bile, cellular extracts, respiratory fluid, lavage fluid, lymph fluid, saliva and other tissue secretions or fluid. Particular biological fluid is whole blood, blood plasma and serum. The biological sample may, therefore, be a fluid-based sample or cells including cells captured to solid support. It is not necessary for a biological sample to be physically removed from a subject, although removal and subsequent analysis of biomarkers in a biological sample is the most convenient method for conducting the instant methods. The biological fluid may undergo an enrichment process or high abundance molecules which might interfere in the assay may be removed.

The present invention is predicated in part on the identification of biomarkers, the collective expression of 2 or more of which, is instructive of TNFα associated T2D as well as complications associated with TNFα associated T2D, such as obesity, blindness, nephropathy and/or cardiovascular disease or the probability of developing TNFα associated T2D.

Reference to “identification” includes ranking, stratifying, or profiling selected 2 or more biomarkers indicative of insulin resistance/sensitivity, or a complication arising therefrom. The ranking, stratifying and profiling are all encompassed by the term “expression signature”.

The present invention extends to derivatives and homologs of the genes in the GES or corresponding PES. Hence, the biomarkers of the present invention include those listed above, as well as genes having nucleotide sequences with 70% identity thereto or capable of hybridizing to the sequence or their complementary forms under high stringency conditions or encoding an amino acid sequence having at least 70% similarity to the amino acid sequence encoded by the genes.

Reference to at least 70% includes 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%.

Particular percentage similarities or identities have at least about 80%, at least about 90%, or at least about 95%.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides and amino acid residues, in length, examples include 12, 13, 14, 15, 16, 17, 18, 19, 20, 21, 22, 23, 24 and 25. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as for example disclosed by Altschul et al. (Nucl Acids Res 25:3389, 1997). A detailed discussion of sequence analysis can be found in Unit 19.3 of Ausubel et al. (“Current Protocols in Molecular Biology” John Wiley & Sons Inc, Chapter 15, 1994-1998).

By “high stringency conditions”, is meant conditions under which the probe specifically hybridizes to a target sequence in an amount that is detectably stronger than non-specific hybridization. High stringency conditions, then, would be conditions which would distinguish a polynucleotide with an exact complementary sequence, or one containing only a few scattered mismatches from a random sequence that happened to have a few small regions (3-10 bases, for example) that matched the probe. Such small regions of complementarity, are more easily melted than a full length complement of 14-17 or more bases and high stringency hybridization makes them easily distinguishable. Relatively high stringency conditions would include, for example, low salt and/or high temperature conditions, such as provided by about 0.02 M to about 0.10 M NaCl or the equivalent, at temperatures of about 50° C. to about 70° C. Such high stringency conditions tolerate little, if any, mismatch between the probe and the template or target strand, and would be particularly suitable for detecting expression of specific biomarkers. It is generally appreciated that conditions can be rendered more stringent by the addition of increasing amounts of formamide.

Reference herein to a high stringency includes and encompasses from at least about 0 to at least about 15% v/v formamide and from at least about 1 M to at least about 2 M salt for hybridization, and at least about 31% v/v to at least about 50% v/v formamide, such as 31, 32, 33, 34, 35, 36, 37, 38, 39, 40, 41, 42, 43, 44, 45, 46, 47, 48, 49 and 50% v/v formamide and from at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for hybridization, and at least about 0.01 M to at least about 0.15 M salt, such as 0.01, 0.02, 0.03, 0.04, 0.05, 0.06, 0.07, 0.08, 0.09, 0.10, 0.11, 0.12, 0.13, 0.14 and 0.15 M for washing conditions. In general, washing is carried out Tₘ=69.3±0.41 (G+C) % (Marmur and Doty, J. Mol. Biol. 5: 109, 1962). However, the Tₙ, of a duplex DNA decreases by 1° C. with every increase of 1% in the number of mismatch base pairs (Bonner and Laskey, Eur J Biochem 46:83, 1974). Formamide is optional in these hybridization conditions. Accordingly, high stringency is defined as 0.1×SSC buffer, 0.1% w/v SDS at a temperature of at least 65° C.

In another embodiment, the present invention provides a method for diagnosing TNFα associated diabetes or a complication arising from TNFα associated diabetes in a subject or a predisposition of a subject to develop TNFα associated diabetes, said method comprising screening for levels of protein or mRNA encoding said protein or a homolog thereof wherein the protein is a biomarker listed in Table 3 in a biological sample from said subject, wherein a difference in the level of the protein of compared to a statistically validated threshold is indicative of TNFα associated diabetes or a complication arising therefrom or a predisposition to develop same.

The expression levels (or protein levels if a PES) provide a statistically validated consistent definition of the biological or physiological state when considered in a group of 2 or more of the 7 biomarkers.

The use of numerical values in the various ranges specified in this application, unless expressly indicated otherwise, are stated as approximations as though the minimum and maximum values within the states ranges were both preceded by the word “about”. In this manner, slight variations above and below the stated ranges can be used to achieve substantially the same results as values within the ranges. Also, the disclosure of these ranges is intended as a continuous range including every value between the minimum and maximum values. In addition, the signature extends to ratios of two or more levels of biomarkers providing a numerical value associated with a level of risk of insulin resistance.

The determination of the levels or concentrations of the 2 or more biomarkers enables establishment of a diagnostic rule based on the application of a statistical and machine learning algorithm. Such an algorithm uses relationships between biomarkers and insulin resistance or sensitivity observed in training data (with known insulin resistance or sensitivity status) to infer relationships which are then used to predict the status of patients with unknown status. An algorithm is employed which provides an index of probability that a patient has TNFα associated a insulin resistance or is developing TNFα associated insulin resistance and therefore TNFα associated T2D.

Hence, the present invention contemplates the use of a knowledge base of training data comprising levels of 2 or more biomarkers as described herein from a subject with TNFα associated insulin resistance to generate an algorithm which, upon input of a second knowledge base of data comprising levels of the same biomarkers from a patient with an unknown insulin resistance status, provides an index of probability that predicts if the insulin resistance or sensitivity is associated with TNFα.

The “subject” is generally a human. However, the present invention extends to veterinary applications. Hence, the subject may also be a non-human mammal such as a bovine, equine, ovine, porcine, canine, feline animal or a non-human primate. Notwithstanding, the present invention is particularly applicable to detecting TNFα associated T2D in a human. Reference to “TNFα associated T2D” includes the spectrum of T2D conditions encompassed by the term “T2D” or “Type 2 diabetes”.

The term “training data” includes knowledge of levels of 2 or more biomarkers relative to a control. A “control” includes a comparison to levels of biomarkers in a subject with known insulin resistance or sensitivity or cured of the condition or may be a statistically determined level based on trials. The term “levels” also encompasses ratios of levels of biomarkers.

The “training data” also include the concentration of one or more of PMK2, Skp1a, CD63, STEAP4, ACS1, CS and/or CLU.

The present invention further contemplates a panel of biomarkers for the detection of TNFα associated T2D insulin resistance or sensitivity in a subject, the panel comprising agents which bind specifically to the biomarkers, the biomarkers selected from two or more of PKM2, Skp1a, CD63, STEAP4, ACS1, CS and/or CLU to determine levels of two or more biomarkers and then subjecting the levels to an algorithm generated from a first knowledge base of data comprising the levels of the same biomarkers from a subject of unknown status with respect to the condition wherein the algorithm provides an index of probability of the subject having or not having TNFα associated T2D insulin resistance or sensitivity.

The agents which “specifically bind” to the biomarkers generally include an immunointeractive molecule such as an antibody or hybrid, derivative including a recombinant or modified form thereof or an antigen-binding fragment thereof. The agents may also be a receptor or other ligand. These agents assist in determining the level of the biomarkers.

The present invention, in certain aspects, is directed to the diagnosis or prognosis of TNFα associated T2D or state of TNFα associated T2D insulin resistance or sensitivity, or a complication associated therewith or a predisposition for developing TNFα associated T2D by comparing GES (or corresponding PES) in the biological sample obtained from the subject. The GES or PES may also be compared to a statistically validated threshold. The statistically validated threshold is based upon levels of biomarkers, in comparable samples obtained from a control population, e.g., the general population or a select population of human subjects. For example, the select population may be comprised of apparently healthy subjects. “Apparently healthy”, as used herein, means individual who have not previously had any signs or symptoms indicating the presence of TNFα associated T2D, including one or more of a family history of diabetes, evidence of factors associated with TNFα associated T2D, including one or more of low activity level, poor diet, excess body weight (especially around the waist), over 45 years old, high blood pressure, high blood levels of triglycerides, HDL cholesterol of less than 35, previously identified impaired glucose tolerance by doctor, previous diabetes during pregnancy or baby weighing more than nine pounds. Apparently healthy individuals also do not otherwise exhibit symptoms of disease. In other words, such individuals, if examined by a medical professional, would be characterized as healthy and free of symptoms of disease. Hence, the control values selected may take into account the category into which the test subject falls. Appropriate categories can be selected with no more than routine experimentation by those of ordinary skill in the art.

The statistically validated threshold is related to the value used to characterize the level of the biomarker, be it a nucleic acid or polypeptide obtained from the subject. Thus, if the level of the biomarker nucleotide or polypeptide is an absolute value, such as the number of copies of a particular transcript or level of a protein per ml of blood, or cell number then the control value is also based upon the number of copies of a particular transcriptor level of a protein per ml of blood, or cell number.

The statistically validated threshold can take a variety of forms. The statistically validated threshold can be a single cut-off value, such as a median or mean. The statistically validated threshold can be established based upon comparative groups such as where the risk in one defined group is double the risk in another defined group. The statistically validated threshold can be divided equally (or unequally) into groups, such as a low risk group, a medium risk group and a high-risk group, or into quadrants, the lowest quadrant being individuals with the lowest risk the highest quadrant being individuals with the highest risk, and the subject's risk of having diabetes or a predisposition to develop diabetes can be based upon which group his or her test value falls.

Statistically validated threshold of the biomarkers obtained, such as for example, mean levels, median levels, or “cut-off” levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference. A “cutoff” value can be determined for each biomarker that is assayed.

Levels of each select biomarker nucleic acid (genomic or nucleomic marker) or polypeptide (proteomic marker) in the subject's biological sample may be compared to a single control value or to a range of control values. If the level of the biomarker in the subject's biological sample is different than the statistically validated threshold, the test subject is at greater risk of developing or having TNFα associated T2D or a condition associated with TNFα associated T2D or a predisposition of a subject to develop TNFα associated T2D than individuals with levels comparable to the statistically validated threshold. The extent of the difference between the subject's GES/PES biomarker(s) levels and statistically validated threshold is also useful for characterizing the extent of the risk of TNFα associated T2D insulin resistance or sensitivity and thereby, determining which individuals would most greatly benefit from certain therapies. In those cases, where the statistically validated threshold ranges are divided into a plurality of groups, such as the statistically validated threshold ranges for individuals at high risk, average risk and low risk, the comparison involves determining into which group the subject's level of the relevant risk predictor falls.

The present predictive tests are useful for determining if and when therapeutic agents that are targeted at preventing TNFα associated T2D or for slowing the progression of TNFα associated T2D or for treating a condition associated with TNFα associated T2D should and should not be prescribed for an individual or selected from a test group of compounds. For example, individuals with values of a GES (or PES) different from a statistically validated threshold, or that are in the higher tertile or quartile of a “normal range,” could be identified as those in need of therapeutic intervention with diabetic therapies, life style changes, etc.

In the practice of this embodiment, one may use a nucleic acid segment that is complementary to the full length of the mRNA specific for the biomarkers listed above, or one may use a smaller segment that is complementary to a portion of the mRNA. Such smaller segments may be from about 14, about 15, about 16, about 17, about 18, about 19, about 20, about 21, about 22, about 23, about 24, about 25, about 25, about 30, about 50, about 75, about 100 or even several hundred bases in length and may be contained in larger segments that provide other functions such as promoters, restriction enzyme recognition sites, or other expression or message processing or replication functions. In an embodiment such probes are designed to selectively hybridize to the biomarkers listed above or protein product thereof. Also useful are the use of probes or primers that are designed to selectively hybridize to a nucleic acid segment having a sequence selected from the group consisting of PKM2, Skp1a, CD63, STEAP4, ACS1, CS and/or CLU.

The methods of the present invention may also include determining the amount of hybridized product. Such determination may be by direct detection of a labeled hybridized probe, such as by use of a radioactive, fluorescent or other tag on the probe, or it may be by use of an amplification of a target sequence, and quantification of the amplified product. A useful method of amplification is a reverse transcriptase polymerase chain reaction (RT-PCR) as described herein. In the practice of such a method, amplification may comprise contacting the target ribonucleic acids with a pair of amplification primers designed to amplify mRNA of the biomarkers, or even contacting the ribonucleic acids with a pair of amplification primers designed to amplify a nucleic acid segment comprising the nucleic acid sequence or complement thereof of a sequence selected from the group consisting of PKM2, Skp1a, CD63, STEAP4, ACS1, CS and/or CLU or a complement thereof.

Diagnostic and prognostic methods may be based upon the steps of obtaining a biological sample from a subject or patient, contacting nucleic acids from the biological sample with an isolated nucleic acid segment specific for a biomarker listed for 2 or more of PJM2, Skp1a, CS63, STEAP4, ACS1, CS and/or CLU under conditions effective to allow hybridization of substantially complementary nucleic acids, and detecting, and optionally further characterizing, the hybridized complementary nucleic acids thus formed.

The methods may involve in situ detection of sample nucleic acids located within the cells of the sample. The sample nucleic acids may also be separated from the cell prior to contact. The sample nucleic acids may be DNA or RNA.

A homolog is considered to be a biomarker gene from another animal species. The present invention extends to the homologous gene, as determined by nucleotide sequence and/or amino acid sequences and/or function, from primates, including humans, marmosets, orangutans and gorillas, livestock animals (e.g. cows, sheep, pigs, horses, donkeys), laboratory test animals (e.g. mice, rats, guinea pigs, hamsters, rabbits), companion animals (e.g. cats, dogs) and captured wild animals (e.g. rodents, foxes, deer, kangaroos).

Antibodies are particularly useful as a diagnostic or prognostic tools for determining a PES of TNFα associated T2D.

For example, specific antibodies can be used to screen for biomarker proteins. The latter is important, for example, as a means for screening for levels of one or more of the biomarkers in a cell extract or other biological fluid such as serum, blood, urine or saliva. Techniques for the assays contemplated herein are known in the art and include, for example, sandwich assays and ELISA.

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and Western blotting, dot blotting, FACS analyses, and the like may also be used.

In one exemplary ELISA, antibodies binding to the encoded proteins of the invention are immobilized onto a selected surface exhibiting protein affinity, such as a well in a polystyrene microtiter plate. Then, a test composition suspected of containing the diabetes biomarker antigen, such as a clinical sample, is added to the wells. After binding and washing to remove non-specifically bound immunocomplexes, the bound antigen may be detected. Detection is generally achieved by the addition of a second antibody specific for the target protein, that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the biomarker antigen are immobilized onto the well surface and then contacted with the antibodies of the invention. After binding and washing to remove non-specifically bound immunocomplexes, the bound antigen is detected. Where the initial antibodies are linked to a detectable label, the immunocomplexes may be detected directly. Again, the immunocomplexes may be detected using a second antibody that has binding affinity for the first antibody, with the second antibody being linked to a detectable label.

Another ELISA in which the proteins or peptides are immobilized, involves the use of antibody competition in the detection. In this ELISA, labelled antibodies are added to the wells, allowed to bind to the biomarker protein, and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immunocomplexes.

The present invention also relates to an in vivo method of imaging TNFα associated T2D or pre-clinical manifestations of TNFα associated T2D using monoclonal antibodies directed to proteins in the PES. Specifically, this method involves administering to a subject an imaging-effective amount of a detectably-labeled biomarker monoclonal antibody or fragment thereof and a pharmaceutically effective carrier and detecting the binding of the labeled monoclonal antibody to the diseased, or in the case of up or down regulated marker genes, healthy tissue. The term “in vivo imaging” refers to any method which permits the detection of a labeled monoclonal antibody of the present invention or fragment thereof that specifically binds to a diseased tissue located in the subject's body. An “imaging effective amount” means that the amount of the detectably-labeled monoclonal antibody, or fragment thereof, administered is sufficient to enable detection of binding of the monoclonal antibody or fragment thereof to the diseased tissue, or the binding of the monoclonal antibody or fragment thereof in greater proportion to healthy tissue relative to diseased tissue.

Kits also form part of the present invention as well as drugs identified herein which are useful in the treatment of TNFα associated T2D.

The present invention further provides the use of a GES or corresponding PES herein described in the manufacture of a medicament or diagnostic assay for Type 2 diabetes or for a compound which reduces insulin resistance or promotes insulin sensitivity in a cell.

The present invention is further described by the following non-limiting examples.

### Example 1

### Determination of a GES of Insulin Resistance

Initially, a model of TNFα-induced insulin resistance in 3T3-L1 adipocytes was established as described previously (Sartipy and Loskutoff J Biol Chem 278:52298-52306, 2003). Following exposure of 3T3-L1 adipocytes to 3 ng/ml TNFα for 72 h, insulin resistance was determined by the ability of the cells to take up 2-deoxyglucose (2-DOG) in response to insulin. TNFα caused a 37% decrease in 2-DOG uptake compared with insulin-stimulated vehicle-treated cells (p<0.0001) (Table 1). To reverse the effects of TNFα, post-treatment with 5 mM ASA and 10 μM TGZ was included in the final 24 h of the 72 h TNFα treatment and found to restore the TNFα-impairment (p=0.0053 to TNFα-treated cells). Individually, ASA and TGZ also fully reversed TNFα effects (p=0.0035 to TNFα-treated cells for TNFα plus TGZ and p=0.0011 to TNFα-treated cells for TNFα plus ASA).

Global gene expression was studied under these conditions to identify a GES representing each biological state (“insulin resistant” and “insulin re-sensitized”). Microarray analyses of vehicle-, TNFα-, and TNFα plus ASA and TGZ co-treated 3T3-L1 adipocyte samples were performed using 20 replicate samples per treatment to facilitate statistical estimation of joint predictors. Overall, the expression of 3325 genes was affected by TNFα treatment compared with vehicle-treated adipocytes (nominal p<0.01) using a robust linear model (based on a multivariate t-distribution) with accompanying likelihood ratio test obtained. Of these, the expression of 1022 genes was reversed following treatment with ASA and TGZ (nominal p<0.01). These 1022 genes were subjected to a Bayesian model selection procedure (Blangero et al. Hum Biol 77:541-559, 2005) where models of up to 7 genes were generated to obtain a Bayesian averaged regression equation that served as the GES. One model consisting of 7 genes was found to have a predictive power of 98% to discriminate between the insulin resistant and insulin re-sensitised states was selected to be the TNFα-based GES. These genes were identified to be acyl-CoA synthetase 1 (ACS1), six transmembrane epithelial antigen of the prostate 4 (STEAP4), S-phase kinase associated protein 1A (Skp1a), pyruvate kinase, muscle 2 (PKM2), CD63, citrate synthase (CS) and clusterin (CLU), and display a variety of functions (Table 2). Their DNA microarray expression profile reveal that five of the seven genes were found to have increased expression following TNFα treatment (STEAP4, PKM2, Skp1a, CD63 and CLU) while two genes (ACS1 and CS) had decreased gene expression relative to vehicle treatment (Table 3).

TNFα downregulation and upregulation of ACS1 and STEAP4 mRNA levels, respectively, have been reported previously (Moldes et al. J Biol Chem 276:33938-33946, 2001; Weiner et al. J Biol Chem 266:23525-23528, 1991). To our knowledge, there have been no reports of direct TNFα-regulation of PKM2, Skp1a and CD63 transcription. Each of the 7 genes was significantly different in its level of gene expression between the vehicle- and TNFα-treated states and also between the TNFα- and TNFα plus ASA and TGZ co-treated samples (p<0.005). Four of the genes (STEAP4, PKM2, CD63 and CLU) remained significantly different between vehicle- and TNFα plus ASA and TGZ co-treatments (p<0.002) indicating only partial reversion of TNFα effects by the insulin sensitising agents while ASA and TGZ fully restored ACS1, Skp1a and CS gene expression back to vehicle control levels. The level of gene expression change for each gene in each condition was confirmed by semi-quantitative real time PCR using a smaller number of samples per treatment (Table 3). Again, all genes remained significantly different between vehicle and TNFα treatments or between TNFα- and TNFα plus ASA and TGZ co-treatments (p<0.05).

In order to identify new insulin sensitizing agents, a small molecule library consisting of 1120 high-purity, off-patent chemical compounds was screened using the GES. First, 3T3 μl adipocytes were cultured in fourteen 96-well plates and incubated with 3 ng/ml TNFα for 72 h prior to the addition of 10 μM of each compound in the last 24 h of TNFα treatment. Four vehicle control, four TNFα and two TNFα plus ASA- and TGZ-treated wells serving as controls were included per 96-well plate. The aim of the screen was to identify compounds that caused the expression of the 7 gene GES to most closely resemble the expression levels observed in the insulin re-sensitized cells as this is likely to indicate that these cells have restored insulin sensitivity. Following RNA extraction and cDNA synthesis, gene expression analysis of the 7 gene GES was performed using the MassARRAY (Sequenom, San Diego, Calif.) [Cullinan and Cantor Pharmacogenomics 9:1211-1215, 2008]. Data are represented as a Z-score residual coefficient correlation (Zrcc); a Z-score that is normalised for sample to sample variation and for the relative contribution that each gene makes to the predictive power of the GES. Compounds were ranked based on their Zrcc score highest to lowest, and as an initial proof of concept validation test, the top ranked 30 compounds and 23 randomly chosen, mid-ranked compounds were subjected to 2-DOG uptake assays to determine their potential insulin sensitising effects. 3T3 μl adipocytes were incubated with 25 μM of each compound prior to performing 2-DOG uptake assays in the presence or absence of submaximal amounts of insulin (0.5 nM for 15 min). As a result, 50% and 63% of the top 30 compounds significantly increased glucose uptake by at 0 and 0.5 nM insulin, respectively, compared with 13% and 30% of the mid-range compounds (p<0.03) (Table 4). At a 10 μM dose, a higher percentage of the top 30 compounds also increased glucose transport at 0 and 0.5 nM insulin compared with the mid-range compounds, however, significance was not reached (Table 4). Overall, these data indicate that the GES analysis enriched for compounds with insulin sensitising properties.

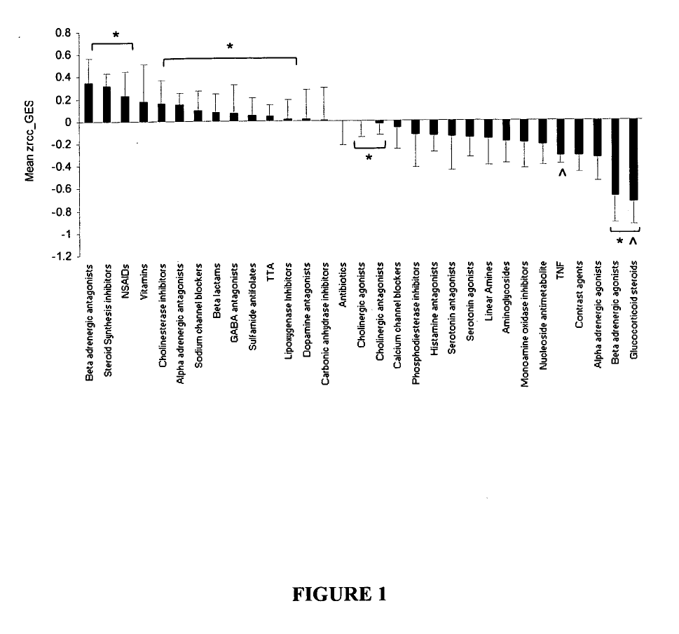

The GES-ranked compounds were next broadly grouped into classes based on known mechanism of action or common structural features and re-represented as the mean Zrcc. Only compound classes with 10 or more members were considered further. As a result, the class of treatments that scored the highest, thus representing the most insulin sensitive cells, were the vehicle-treated cells with an average Zrcc score of 1.76±0.37; (p<0.0001 to TNFα treatment and p<0.002 to TNFαplus ASA and TGZ co-treatments). Thirteen out of the top 20 ranked compound classes, which included the TNFα plus ASA and TGZ co-incubated samples, have significantly increased average Zrcc scores compared with the TNFα treatment (p<0.05) while only glucocorticoids and beta adrenergic agonists scored significantly lower than the TNFα samples (p<0.05) (FIG. 1).

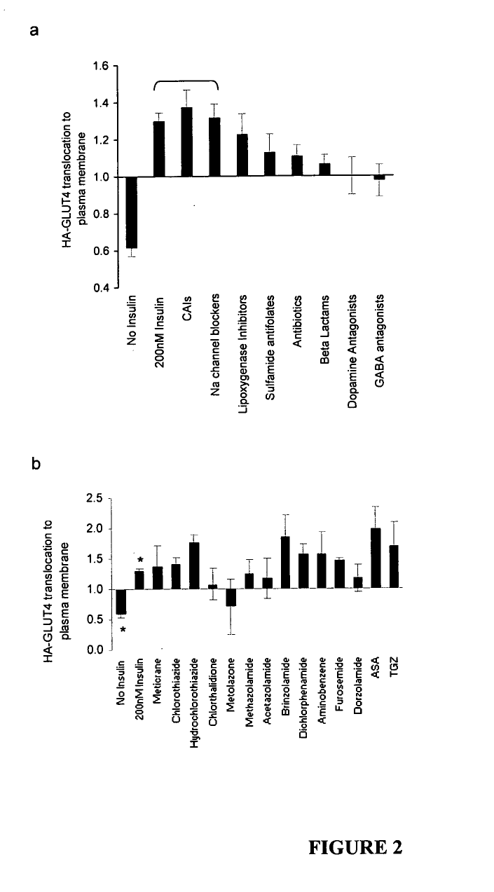

A secondary screen was next undertaken to test the 8 drug classes ranked most similar to the insulin re-sensitised TNFαplus ASA and TGZ co-treated samples to determine their ability to affect exogenous HA-tagged GLUT4 translocation to the plasma membrane in the presence of submaximal 0.5 nM insulin (Govers et al. Mol Cell Biol 24:6456-6466, 2004). Members from the sodium channel blockers, beta lactams, GABA antagonists, sulfamide antifolates, lipoxygenase inhibitors, dopamine antagonists, carbonic anhydrase inhibitors (CAIs) and antibiotics were incubated at 10 μM doses for 20 h and the overall effect of each class member was assessed and averaged. Only the combined members from the CAIs and sodium channel blockers classes were found to significantly increase HA-GLUT4 translocation above submaximal insulin in addition to acute maximal 200 nM insulin (p<0.03) (FIG. 2a). Further investigation of the compounds comprising the sodium channel blockers class revealed that many of them are known to affect glucose and lipid metabolism including quinic acid (Zrcc of 1.673 in the GES screen), procaine (Zrcc=0.312) and disopyramide (Zrcc=0.126) (Boden et al. Circulation 85:2039-2044, 1992; Hope-Gill et al. Horm Metab Res 6:457-463, 1974; Kojima et al. Chem Pharm Bull (Tokyo) 51:1006-1008, 2003; Taketa and Yamamoto Acta Med Okayama 34:289-292, 1980). Therefore, the class of CAIs was the subject of focus for further analysis and it was found that each CAI member, except for metolazone, increased HA-GLUT4 translocation above submaximal insulin effects as well as TGZ and ASA treatment (FIG. 2b).

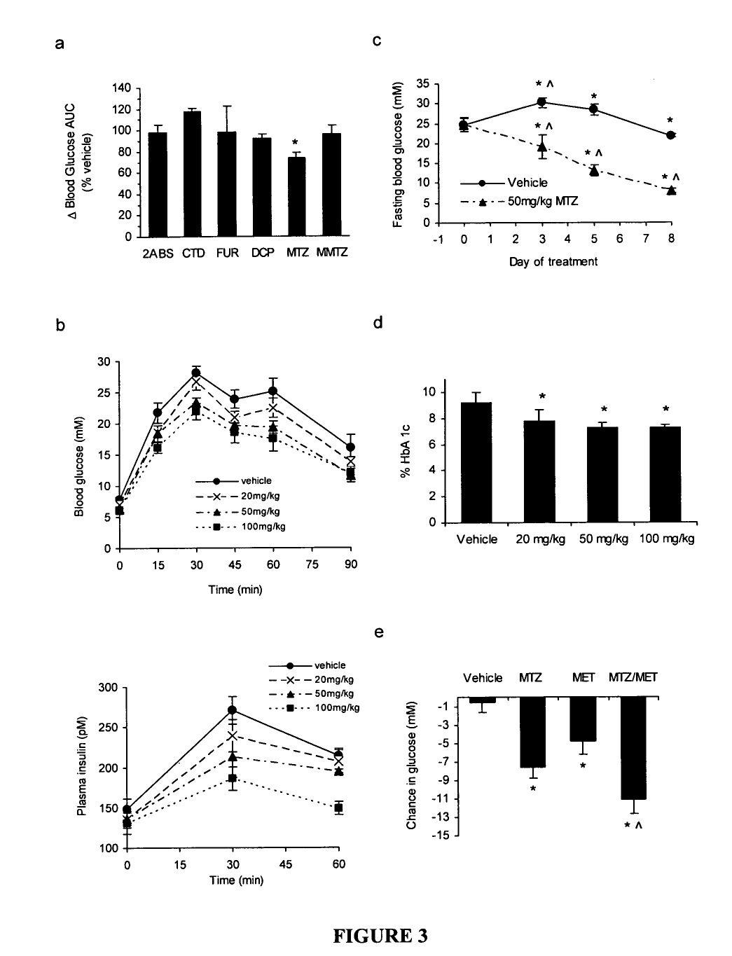

Whether a selection of CAIs exhibited any insulin sensitising effects in vivo was investigated. Diet-induced obese (DIO) mice were treated with each CAI at 50 mg/kg/day for 14 days. The CAIs 2-aminobenzene sulphonamide (2ABS), chlorthalidone (CTD), furosemide (FUR) and dichlorphenamide (DCP), did not affect glucose disposal in DIO mice (FIG. 3a). On the other hand, methazolamide (MTZ) elicited a 27% reduction in the incremental area under the glucose curve (AUC) compared with vehicle-treated animals (p<0.03). This phenotype was not observed when DIO mice were treated with an N-methylated derivative of methazolamide (MMTZ) (FIG. 3a). These derivatives have one of the amine hydrogens responsible for CA inhibition replaced with a methyl group to prevent binding to CA (Relman et al. J Clin Invest 39:1551-1559, 1960) and typically exhibit 100 times less carbonic anhydrase inhibitory activity in vitro. The effects of MTZ in circulating glucose levels were achieved without significant changes in body weight, food and water intake or epididymal fat mass compared with vehicle-treated mice. Furthermore, 24 h urine output and creatine excretion were not significantly altered between MTZ- and vehicle-treated mice indicating that the difference observed in glucose metabolism in these animals was not due to a diuretic effect of MTZ. Additional studies in DIO mice treated with 2ABS, CTD, FUR or DCP found no significant effect on glucose tolerance at a concentration range of 20-100 mg/kg/day for 14 days. MTZ treatment caused a significant reduction in circulating glucose levels at concentrations above 20 mg/kg compared with vehicle-treated mice (upper panel, FIG. 3b). The hypoglycaemic effect was accompanied with a dose-dependent decrease in plasma insulin levels (lower panel, FIG. 3b).

MTZ in vivo efficacy in db/db mice, an animal model of type 2 diabetes, was next determined. Mice treated with 20 or 50 mg/kg/d MTZ for 14 days had a dose-dependent decrease in fasting glucose levels (p<0.05; FIG. 3c). This effect was seen after 3 days of MTZ administration and peaked after 7 days. No change in body weight, food or water intake was observed in vehicle-treated and 20 mg/kg MTZ-treated animals. However, after 7 days of treatment with 50 mg/kg MTZ, a 4% reduction in body weight (38.6±1.0 vs 37.0±1.0 g vehicle- vs MTZ-treated animals, respectively; p<0.001) and a 27% decrease in food intake (6.3±0.3 vs 4.8±0.5 g/day vehicle- vs MTZ-treated animals, respectively; p<0.01) was observed. To investigate whether the decrease in glycemia caused by MTZ was due to reduced food intake and loss in body weight, changes in blood glucose were monitored in pair-fed vehicle and MTZ-treated db/db mice for 8 days. This resulted in an 8% reduction in body weight in vehicle-treated mice (41.7±2.7 g day 0 versus 38.3±3.2 g day 8, n=6/group; p<0.001). However, only MTZ-treated animals displayed significantly lowered fasting blood glucose after the treatment period (0.6±1.9 mM pair-fed vehicle versus −6.2±1.5 mM MTZ-treated; p<0.02). These results indicate that the hypoglycaemic effects of MTZ were not due to reductions in food intake or body weight loss. Furthermore, the antidiabetic effects of MTZ in db/db mice were not due to increased loss of glucose by urinary secretion. Mice treated with MTZ (50 mg/kg/d) for 14 d were placed into metabolic cages for 24 h. MTZ treatment significantly reduced glucose urine concentration by 18% compared with vehicle-treated mice (p<0.03) while no significant differences in total urine glucose excretion, urine volume and water intake were observed. The effect of MTZ treatment on HbA1c levels in db/db mice was next examined. It was found that treatment with MTZ resulted in up to 23% lower HbA1c levels (p<0.003) (FIG. 3d). The combined effect of MTZ with other known insulin sensitizing therapeutic agents was also investigated. db/db mice treated with 300 mg/kg metformin or 20 mg/kg MTZ for 8 d both exhibited a significantly lower change in blood glucose levels compared with vehicle-treated animals (p<0.05) (FIG. 3e). Co-administration of metformin and MTZ caused a further blood glucose lowering effect to that of metformin alone (p<0.02).

### Example 2

### Characterization of an Insulin Resistant Population In Vivo Using the TNFα-Based GES

The biological relevance in vivo of the in vitro generated TNFα-based GES was tested. A global human gene expression data set was used to evaluate whether the GES could characterize insulin resistant phenotypes in humans. This profiling was undertaken on lymphocytes as part of the San Antonio Family Heart Study (Blangero Nat Genetics 2007) and mapped the expression of 47,289 transcripts in 1,240 individuals from 42 extended family pedigrees using Illumina bead-based technology. The frequency of diabetes in this population was 15.4%. The characteristics of the subjects were as follows (mean±SD): Age 39.3±16.7 y, BMI 29.3±6.6 kg/m², fasting glucose 100.6±43.8 mg/dl, fasting insulin 16.2±19.1.

This dataset also includes anthropometric measurements (such as BMI and other body composition measures), insulin sensitivity measures (oral glucose tolerance test) and standard blood chemistry parameters including plasma glucose, insulin, lipids and cytokine levels. Using this dataset we detected the 7-gene GES identified from 3T3-L1 adipocytes in the human expression profiling dataset and calculated an aggregate GES score comprising the sum of the absolute values of the standardized expression units of each of these 7 genes taking into account the direction of change. A higher level of insulin resistance as measured by Homa_IR (homeostasis model of assessment for insulin resistance based on insulin and glucose) was observed in subjects with a high GES score (Spearmans rho 0.138; p=0.0000012). After normalization for the effects of age and sex, statistically significant differences were observed between the highest and lowest quartiles of subjects based on GES score (n>400 in each quartile) for fasting plasma insulin (p=0.0004), BMI (p=0.00000044), triglyceride levels (p=0.001) and Homa_IR (p=0.00081; Table 5). These observations are consistent with the GES characterizing the most insulin resistant subgroup in this study population.

TABLE 1
Reversal of TNFα-induced insulin resistance in 3T3-L1 adipocytes

| | 2-DOG | p value | p value | |
|---|---|---|---|---|
| | Uptake | compared | compared | |
| | (% of insulin- | with insulin- | with TNFα- | |
| | stimulated | stimulated | treated, insulin- | |
| Treatment | alone) | cells | stimulated cells, | n |
| Acute Insulin | 100.0 ± 9.7% | — | 1.52 × 10⁻⁷ | 9 |
| (Ins; 10 nM, 30’) | | | | |
| T + Ins | 65.5 ± 3.9% | 1.52 × 10⁻⁷ | — | 9 |
| (T; 3 ng/ml | | | | |
| TNFα, 72 h + Ins) | | | | |
| TTA + Ins | 91.3 ± 4.5% | NS | 0.0053 | 6 |
| (T + 10 μM TGZ | | | | |
| & 5 mM ASA, | | | | |
| final 24 h + Ins) | | | | |

3T3-L1 adipocytes were either treated with vehicle—(Veh), 3 ng/ml TNFα-(TNF) or 3 ng/ml TNFα plus 10 μM TGZ and 5 mM ASA (TTA) as detailed above. Cells were then treated with insulin (0 or 10 nM) for 30 min followed by measurement of 2-deoxyglucose (2-DOG) uptake over the final 10 min of insulin stimulation. Data are presented as % change in 2-DOG uptake compared with vehicle-treated, insulin-stimulated cells and represent mean values±SEM of >3 independent experiments and each data point was assayed in triplicate. The fold increase in 2-DOG uptake for the vehicle-treated, insulin-stimulated adipocytes above basal level was 6.6±0.6 (p=1.77×10⁻⁷ compared with vehicle-treated alone). The amount of 2-DOG transported in vehicle-treated adipocytes was 20.5±3.4 pmol/min/well. Statistical analyses were performed using Student's t-Test assuming 2-tailed distribution and 2-sample equal variance.

TABLE 2
Identity of the 7 genes comprising the TNFα-based GES.

| | | NCBI | |
|---|---|---|---|
| Gene names | | reference no. | Proposed function |
| ACS1/FACL2/ | /palmitoyl-CoA | NM_007981 | Fatty acid transport |
| ligase | | | and metabolism |
| | | | {Soupene, 2008 #25} |
| CD63 | | NM_007653 | Cell adhesion |
| | | | and motility |
| | | | {Maecker, 1997 #26} |
| STEAP4/TIARP/STAMP2 | | NM_054098 | Iron/copper reductase; |
| | | | regulator of |
| | | | metabolic homeostasis |
| | | | {Ohgami, 2006 #27; |
| | | | Wellen, 2007 #40} |
| Skp1a | | NM 011543 | Pro-ubiquination; cell |
| | | | cycle regulator |
| | | | {Peters, 1998 #28} |
| PKM2 | | NM_011099 | Aerobic glycolysis |
| | | | and tumorigenesis |
| | | | {Christofk, 2008 #29} |
| CS | | NM_026444 | Citric acid cycle |
| | | | {Goldenthal et al, 1998} |
| CLU | | NM_013492 | Pro-and anti- |
| | | | apoptotic factor |
| | | | {Han et al, 2001; |
| | | | Zhang et al, 2005} |

Abbreviations:
ACS1, acyl-CoA synthetase long-chain family member 1/FACL2, fatty-acid-Coenzyme A ligase, long-chain 2;
STEAP4, six transmembrane epithelial antigen of the prostate/TIARP, TNFα-induced adipose-related protein/STAMP2, six transmembrane protein of prostate 2;
Skp1a, S-phase kinase associated protein 1A;
Pkm2, pyruvate kinase, muscle 2;
CD63, CD63 antigen/Melanoma-associated antigen MLA1/Melanoma-associated antigen ME491/Granulophysincs;
CS, citrate synthase and CLU, Clusterin/Apolipoprotein J/mouse sulfated glycoprotein-2 (MSGP-2).

TABLE 3
Expression profiling of the TNFα-based GES.

| | Gene Expression Levels | | | |
|---|---|---|---|---|
| | (normalised to Vehicle-treated cells set at ‘1’) | | | |
| | Microarray | | RT-PCR | |
| Gene | TNF | TTA | TNF | TTA |
| STEAP4 | 2.74 ± 0.22% | 1.77 ± 0.17% | 6.59 ± 0.49% | 3.31 ± 0.80% |
| | *p = 0.0000 | *p = 0.0010 | *p = | *p = 0.0232 |
| | | \hat{ }p = 0.0035 | 4.92 × 10⁻⁶ | \hat{ }p = 0.0083 |
| PKM2 | 1.95 ± 0.14% | 1.46 ± 0.07% | 2.15 ± 0.18% | 1.26 ± 0.19% |
| | *p = 0.0000 | *p = 0.0000 | *p = 0.0017 | *NS |
| | | \hat{ }p = 0.0001 | | \hat{ }p = 0.0156 |
| ACS1 | 0.41 ± 0.06% | 0.98 ± 0.12% | 0.36 ± 0.07% | 0.81 ± 0.15% |
| | *p = 0.0000 | *NS | *p = 0.0003 | *NS |
| | | \hat{ }p = 0.0000 | | \hat{ }p = 0.0268 |
| Skp1a | 1.36 ± 0.04% | 0.94 ± 0.03% | 1.58 ± 0.18% | 0.77 ± 0.09% |
| | *p = 0.0000 | *NS | *p = 0.0168 | *p = 0.0519 |
| | | \hat{ }p = 0.0000 | | \hat{ }p = 0.0081 |
| CD63 | 1.48 ± 0.05% | 1.14 ± 0.05% | 1.56 ± 0.20% | 1.06 ± 0.11% |
| | *p = 0.0000 | *p = 0.0080 | *p = 0.0454 | *NS |
| | | \hat{ }p = 0.0000 | | \hat{ }p = 0.0487 |
| CS | 0.73 ± 0.02% | 1.05 ± 0.03% | 0.54 ± 0.05% | 0.93 ± 0.12% |
| | *p = 0.0000 | *NS | *p = 0.0195 | *NS |
| | | \hat{ }p = 0.0040 | | \hat{ }p = 0.0621/NS |
| CLU | 2.72 ± 0.03% | 1.74 ± 0.06% | 7.76 ± 0.76% | 3.17 ± 0.33% |
| | *p = 0.0000 | *p = 0.0000 | *p = 0.00002 | *p = 0.0005 |
| | | \hat{ }p = 0.0000 | | \hat{ }p = 0.0015 |

Microarray expression profiling (a) and real time PCR analysis (b) of the 7 gene GES in vehicle-treated (Veh), TNFα-treated (TNF) and TNFα plus TGZ and ASA co-treated (TTA) 3T3-L1 adipocytes. The 7 genes are acyl-CoA synthetase 1 (ACS1), six transmembrane epithelial antigen of the prostate 4 (STEAP4), S-phase kinase associated protein 1A (Skp1a), pyruvate kinase, muscle 2 (PKM2), CD63 antigen (CD63), citrate synthase (CS) and clusterin (CLU). Gene expression values are normalised to vehicle-treated values (represented as ‘1’). Data are represented as mean values±SEM; n=20 (a) or n=5 (b) per sample. *p<0.05 to vehicle-treated and ̂p<0.05 to TNFα-treated samples. Statistical analyses were performed using Student's t-Test assuming 2-tailed distribution and 2-sample equal variance.

TABLE 4
Ranking of compound families by the TNFα-based GES

| | mean | Sample | p value | p value |
|---|---|---|---|---|
| Drug Class | Zrcc ± SEM | no | to TNF | to TTA |
| Vehicle-treated | 1.76 + 0.37 | 55 | 2.0 × 10⁻⁷ | 0.001 |
| Beta adrenergic | 0.35 + 0.22 | 17 | 2.0 × 10⁻⁴ | 0.059 |
| antagonists | | | | |
| Steroid Synthesis | 0.32 + 0.11 | 11 | 2.0 × 10⁻⁴ | 0.111 |
| inhibitors | | | | |
| NSAIDs | 0.23 + 0.22 | 38 | 0.007 | 0.473 |
| Vitamins | 0.18 + 0.33 | 11 | 0.051 | 0.627 |
| Cholinesterase | 0.16 + 0.21 | 13 | 0.009 | 0.576 |
| inhibitors | | | | |
| Alpha adrenergic | 0.15 + 0.10 | 18 | 0.001 | 0.458 |
| antagonists | | | | |
| Sodium channel | 0.10 + 0.17 | 29 | 0.010 | 0.787 |
| blockers | | | | |
| Beta lactams | 0.08 + 0.15 | 26 | 0.008 | 0.826 |
| GABA antagonists | 0.07 + 0.26 | 13 | 0.046 | 0.916 |
| Sulfamide antifolates | 0.05 + 0.15 | 20 | 0.014 | 0.959 |
| TTA | 0.04 + 0.10 | 29 | 0.003 | 1.000 |
| Lipoxygenase | 0.02 + 0.17 | 16 | 0.036 | 0.891 |
| Inhibitors | | | | |
| Dopamine antagonists | 0.01 + 0.26 | 11 | 0.092 | 0.898 |
| Carbonic anhydrase | 0.01 + 0.29 | 11 | 0.111 | 0.880 |
| inhibitors | | | | |
| Antibiotics | 0.00 + 0.21 | 13 | 0.079 | 0.821 |
| Cholinergic agonists | 0.00 + 0.14 | 19 | 0.032 | 0.775 |
| Cholinergic | −0.03 + 0.10 | 62 | 0.019 | 0.654 |
| antagonists | | | | |
| Calcium channel | −0.06 + 0.20 | 20 | 0.129 | 0.614 |
| blockers | | | | |
| Phosphodiesterase | −0.13 + 0.29 | 12 | 0.319 | 0.466 |
| inhibitors | | | | |
| Histamine antagonists | −0.14 + 0.14 | 34 | 0.207 | 0.293 |
| Serotonin antagonists | −0.15 + 0.29 | 14 | 0.402 | 0.430 |
| Serotonin agonists | −0.15 + 0.17 | 15 | 0.309 | 0.296 |
| Linear Amines | −0.16 + 0.23 | 10 | 0.421 | 0.340 |
| Aminoglycosides | −0.19 + 0.19 | 26 | 0.439 | 0.256 |
| Monoamine oxidase | −0.20 + 0.23 | 14 | 0.511 | 0.258 |
| inhibitors | | | | |
| Nucleoside | −0.22 + 0.18 | 13 | 0.563 | 0.169 |
| antimetabolite | | | | |
| TNF | −0.32 + 0.07 | 57 | 1.000 | 0.003 |
| Contrast agents | −0.32 + 0.14 | 11 | 0.987 | 0.052 |
| Alpha adrenergic | −0.33 + 0.21 | 13 | 0.924 | 0.070 |
| agonists | | | | |
| Beta adrenergic | −0.68 + 0.24 | 13 | 0.045 | 0.001 |
| agonists | | | | |
| Glucocorticoid | −0.74 + 0.19 | 20 | 0.012 | 3.0 × 10⁻⁴ |
| steroids | | | | |

TABLE 5
Correlation of the 7-gene TNFα-based GES with quantitative traits.

| | Pearson | Significance | |
|---|---|---|---|
| Quantitative Trait | Correlation | (2-tailed) | N |
| Age | −0.044 | 0.125 | 1240 |
| Normalised Fasting Glucose | −0.053 | 0.085 | 1051 |
| Normalised Fasting Insulin | −0.139** | <0.001 | 1035 |
| Normalised BMI | −0.137** | <0.001 | 1224 |
| Normalised HOMA_IR score | −0.109** | <0.001 | 1223 |
| Normalised HOMA_IR score | −0.137** | <0.001 | 1035 |
| (Non-diabetics only) | | | |
| HDL Cholesterol, normalised | −0.001 | 0.982 | 1192 |
| Triglycerides, normalised | −0.072* | 0.014 | 1160 |
| % Body Fat, normalised | −0.114** | <0.001 | 1035 |

**Correlation is significant at the 0.01 level (2-tailed) and
*at the 0.05 level (2-tailed).

Hence, The GES significantly identified obese individuals with high levels of insulin who have increased insulin resistance according to the Homa IR index (driven by insulin). Standardization of the GES score with Age lowered the strength of the association, but in all cases remained significantly different except for total body fat.

### Example 3

### Comparison of the TNFα 7 Gene GES Versus Single Gene GES

This example highlights the efficacy of a 7 gene GES versus single gene candidates to screen for compounds with insulin sensitizing properties in contrast to compounds known to impair insulin action in vitro and in vivo. The 7 genes were PM2, Skp1a, CD63, STEAP4, ACS1 (FACL2), CS and CLU. The single genes selected for comparative purposes were ACS1 (FACL2), CD63, PKM2 and Skp1a.

The 7-gene TNFα-based GES was able to significantly characterize the insulin re-sensitized (TTA) adipocytes with a positive Zrcc score of 0.5±0.2 from the insulin-resistant (TNF) adipocytes with a negative score of −0.6±0.1 (p=2.9×10⁻⁷) [Table 6]. The data in Table 6 demonstrate that if the screen was performed with only ACS1 (FACL2), CD63, PKM2 or Skp1a, such a single gene screen would not distinguish the insulin re-sensitized (TTA) from the insulin resistant (TNF) cells. In a CD63-only screen, TTA results with a negative score and TNF with a positive (p=0.02). Serving as internal controls in the screen, 3T3-L1 adipocytes were either treated with vehicle (0.2% w/v DMSO) for 72 h (n=55), 3 ng/ml TNFα for 72 h (TNF (n=57) or 3 ng/ml TNFα for 72 h plus 10 μM TGZ and 5 mM ASA in the final 24 h of the TNFα incubation (TTA) (n=29). The gene expression analysis of the GES was performed using the Mass ARRAY system (Cullinan and Cantor, 2008 supra). Data are calculated as a Z-score of the residual component following adjustment for total expression levels and incorporating the correlation coefficients derived from the Bayesian prediction model (Zrcc). The resulting metric is a Z-score that was normalized for sample to sample variation and for the relative contribution that each gene makes to the predictive power of the GES. Data are represented as mea Zrcc values±SEM (with p values compared with TNF-treated sample). The statistical analyses were performed using Student's t-Test assuming 2-tailed distribution and 2-sample equal variance.

TABLE 6
Ranking of insulin re-sensitized (TTA) and
the insulin resistant (TNF) cells by the 7
gene versus single gene TNFα GES.

| | Mean Zrcc_GES ± SEM | | |
|---|---|---|---|
| | TTA | TNF | p value |
| 7-gene | 0.5 ± 0.2 | −0.6 ± 0.1 | 2.9 × 10⁻⁷ |
| FACL2 | −0.1 ± 0.1 | −0.1 ± 0.03 | NS |
| CD63 | −0.1 ± 0.1 | 0.1 ± 0.05 | 0.02 |
| PKM2 | −0.2 ± 0.1 | −0.2 ± 0.04 | NS |
| Skp1a | 0.1 ± 0.04 | −0.1 ± 0.1 | NS |

Known insulin sensitizers such as the monoamine oxidase inhibitor furazoldone (FUR), NSAIDs mesalamine (MES) and fosfosal (FOS), and estrogen (EST) were used to establish the dynamic range and confidence in the GES screen with all compounds scoring a positive Zrcc. In contrast, known insulin resistance-inducing compounds such as the glucose uptake inhibitor ajmaline (AJM) and the glucocorticoid corticosterone (COR) further validated the screen scoring a negative Zrcc. Within these screening validation parameters, any compound scoring a positive Zrcc and within the similar range of TTC was considered as a potential insulin sensitizing compound and was taken into secondary screens. VVP808 was identified as a compound with insulin sensitizing action using these parameters (Table 7). A CS- or STEAP4-only screen would have distinguished TTA- from TNF-treated cells, however, with a narrow dynamic range that would not be sufficient to differentiate between compounds that are TTA- versus TNF-like (CS:0.1±0.1 for TTA versus −0.05±0.03 for TNF, p=0.001; STEAP4: 0.2±0.05 for TTA versus −0.02±0.05 for TNF, p=0.005). See Table 7. A CS- or STEAP4-only screen would have also failed the validation parameters with furazolidone and fosfosal or mesalamine and ajmaline scoring as false negatives or positives. A CLU-only screen would have distinguished TTA- from TNF-treated cells (0.3±0.1 for TTA versus −0.3±0.1 for TNF, p=2.9×10⁻⁷). However, using only CLU to screen the compound library would have failed to select VVP808 as a potential insulin sensitizing agent (false negative).

In this assay, 3T3-L1 adipocytes were incubated with TNFα for 72 h followed by the addition of 10 μM of each compound in the final 24 h of the TNFα treatment (n=2). Compounds include VVP808, published insulin sensitizers furazolidone (FUR) (monoamine oxidase inhibitor), mesalamine (MES) and fosfosal (FOS) (NSAIDs), and estradiol-17-beta (EST), and known insulin resistance-inducing compounds such as ajmaline (AJM) (glucose uptake inhibitor) and corticosterone (COR) (glucocorticoid). Serving as internal controls in the screen, 3T3-L1 adipocytes were either treated with vehicle (0.2% DMSO) for 72 h (n=55), 3 ng/ml TNFα for 72 h) TNF) (n=57) or 3 ng/ml TNFα for 72 h plus 10 μM TGZ and 5 nM ASA in the final 24 h of the TNFα incubation (TTA) (n=29). The gene expression analysis of the GES and calculation of the Z-score was performed as detailed above. Data are represented as mean Zrcc values±SEM. The results are presented in Table 7.

TABLE 7
Ranking of VVP808 and known insulin sensitizing or resistance-inducing compounds
by the 7 gene versus single gene TNFα-based GES

| | Mean Zrcc_GES ± SEM | | | | | | | | |
|---|---|---|---|---|---|---|---|---|---|
| | TTA | 808 | FUR | MES | FOS | EST | TNF | AJM | COR |
| 7-gene | 0.5 ± 0.2 | 0.6 | 0.8 | 4.9 | 2.0 | 0.2 | −0.6 ± 0.1 | −0.5 | −2.7 |
| CS | 0.1 ± 0.1 | 0.2 | −0.2 | 3.1 | −0.05 | 0.04 | −0.05 ± 0.03 | −0.5 | −0.5 |
| STEAP4 | 0.2 ± 0.05 | 0.5 | 0.7 | −0.4 | 0.3 | 0.3 | −0.02 ± 0.05 | 0.6 | −0.2 |
| CLU | 0.3 ± 0.1 | −0.4 | 0.4 | −0.9 | 0.6 | 0.5 | −0.3 ± 0.1 | −0.2 | 0.5 |

### Example 4

### Optimization of Type 2 Diabetes Treatment

A GES is used to classify patients with diabetes, so that their treatment can be optimized by using compounds identified using the same or similar GES. This approach is supported by data in Example 2 (Table 5) showing that a GES can be used to identify a sub-group of patients with increased obesity and insulin resistance. These patients are proposed to benefit from treatment with drugs that identified using the same GES.

Monitoring GES's over a course of treatment may result in changing signatures due to reversal of the effects of fatty acids or glucocorticoids, for example, and these different GES's are measured in patients. The treatments are then optimized according to which GES they most closely represent.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Alizadeh et al. Nature 403:503-511, 2000
Altschul et al. Nucl Acids Res 25:3389, 1997.
Arany et al. Proc Natl Acad Sci USA 105:4721-4726, 2008
Ausubel et al. (“Current Protocols in Molecular Biology” John Wiley & Sons Inc, Chapter 15, 1994-1998
Blangero et al. Hum Biol 77:541-559, 2005
Blangero Nat Genetics 2007
Boden et al. Circulation 85:2039-2044, 1992
Bonner and Laskey, Eur J Biochem 46:83, 1974
Cooper et al. Nat Clin Pract Urol 4:677-687, 2007
Cullinan and Cantor Pharmacogenomics 9:1211-1215, 2008
Gao et al. J Biol Chem 278:24944-24950, 2003
Govers et al. Mol Cell Biol 24:6456-6466, 2004
Hope-Gill et al. Horm Metab Res 6:457-463, 1974
Hotamisligil, Nature 444: 860-867, 2006
Knapp, R. G., and Miller, M. C. (1992). Clinical Epidemiology and Biostatistics. William and Wilkins, Harual Publishing Co. Malvern, Pa.
Kojima et al. Chem Pharm Bull (Tokyo) 51:1006-1008, 2003
Marmur and Doty, J. Mol. Biol. 5: 109, 1962
Miles et al. Diabetes 46:1678-1683, 1997
Mlinar, et al. Clin Chim Acta 375-20-35, 2007
Moldes et al. J Biol Chem 276:33938-33946, 2001
Nuyten and van de Vijver Semin Radiat Oncol 18:105-114, 2008
Ohsumi et al. Endocrinology 135:2279-2282, 1994
Peraldi and Spiegelman J Clin Invest 100:1863-1869, 1997
Qi and Pekala Proc Soc Exp Biol Med 223:128-135, 2000
Reiman et al. J Clin Invest 39:1551-1559, 1960
Ruan and Lodish Cytokine Growth Factor Rev 14:447-455, 2003
Sartipy and Loskutoff J Biol Chem 278:52298-52306, 2003
Taketa and Yamamoto Acta Med Okayama 34:289-292, 1980
Tang et al. Proc Natl Acad Sci USA 103:2087-2092, 2006 van de Vijver et al. N Engl J Med 347:1999-2009, 2002
Weiner et al. J Biol Chem 266:23525-23528, 1991
Yuan et al. Science 293:1673-1677, 2001

1. A gene expression signature (GES) or corresponding proteomic expression signature (PES) indicative of Type 2 diabetes or symptoms thereof, said GES or PES comprising expression levels of at least two genes or gene products selected from the list comprising PKM2, Skp1a, CD63, STEAP4, ACS1 (FACL2), CS and CLU.

2. (canceled)

3. (canceled)

4. (canceled)

5. (canceled)

6. (canceled)

7. (canceled)

8. The GES or PES of claim 1 wherein the Type 2 diabetes is TNFα associated Type 2 diabetes.

9. The GES or PES of claim 1 wherein a state of insulin sensitivity is indicated by a decrease in expression of PKM2, Skp1a, CD63, STEAP4 and CLU relative to a control.

10. The GES or PES of claim 1 wherein a state of insulin sensitivity is indicated by an increase in expression of ACS1 (FACL2) and CS relative to a control.

11. A method for the diagnosis or prognosis of Type 2 diabetes or a predisposition for the development of Type 2 diabetes or a complication associated with Type 2 diabetes in a subject, said method comprising: (a) obtaining a biological sample from a subject; (b) determining the GES or corresponding PES based on 2 or more of PKM2, Skp1a, CD63, STEAP4, ACS1, CS and/or CLU in the biological sample; and (c) comparing the GES in the biological sample to a statistically validated threshold, wherein the GES or its corresponding PES is instructive of the level of insulin sensitivity or resistance.

12. (canceled)

13. (canceled)

14. (canceled)

15. (canceled)

16. (canceled)

17. (canceled)

18. The method of claim 11 wherein the Type 2 diabetes is TNFα associated Type 2 diabetes.

19. The method of claim 11 wherein a state of insulin sensitivity is indicated by a decrease in expression of PKM2, Skp1a, CD63, STEAP4 and CLU relative to a control.

20. The method of claim 11 wherein a state of insulin sensitivity is indicated by an increase in the expression of ACS1 (FACL2) and CS relative to a control.

21. A method for identifying a compound which reduces the level of insulin resistance in cells, said method comprising contacting insulin resistant cells having a first GES or corresponding PES which is instructive of insulin resistance (first knowledge base) and then screening for a second GES or corresponding PES which is instructive of insulin sensitivity (second knowledge base) wherein a compound which promotes development of the second GES is selected as the compound.

22. The method of claim 21 wherein the GES or corresponding PES comprises from at least two to seven genes or gene products selected from the listing comprising PKM2, Skp1a, CD63, STEAP4, ACS1 (FACL2), CS and CLU.

23. (canceled)

24. (canceled)

25. (canceled)

26. (canceled)

27. (canceled)

28. The method of claim 21 wherein the insulin resistance is associated with Type 2 diabetes.

29. The method of claim 28 wherein the Type 2 diabetes is TNFα associated Type 2 diabetes.

30. The method of claim 21 wherein a state of insulin sensitivity is indicated by a decrease in expression of PKM2, Skp1a, CD63, STEAP4 and CLU relative to a control.

31. The method of claim 21 wherein a state of insulin sensitivity is indicated by an increase of expression of ACS1 (FACL2) and CS relative to a control.

32. (canceled)

33. (canceled)

34. (canceled)

35. A method for stratifying a subject in need of treatment for Type 2 diabetes to facilitate therapeutic intervention, said method comprising determining a GES of corresponding PES according to claim 1 for the subject and selecting a medicament identified as a diabetes symptom reversing agent using the same or substantially similar GES or corresponding PES to the GES or PES used to stratify the subject.

36. A method of treatment of a subject with Type 2 diabetes or symptoms thereof, said method comprising determining the a GES or corresponding PES according to claim 1 for the subject and administering a medicament identified as a diabetes symptom reversing agent using the same or substantially similar GES or corresponding PES to the GES or PES determined on said subject.

37. A method of treatment of a subject with Type 2 diabetes or symptoms thereof, said method comprising determining the a GES or corresponding PES according to claim 1 for the subject and administering a medicament identified as a diabetes symptom reversing agent using the same or substantially similar GES or corresponding PES to the GES or PES determined on said subject and monitoring the GES or corresponding PES over time and adjusting the medication such that the medicament has a GES or corresponding PES the same or substantially similar to the last determined GES or PES for the subject.

38. (canceled)

39. (canceled)

40. (canceled)

41. (canceled)

42. (canceled)

43. (canceled)

44. The method of claim 35 wherein the Type 2 diabetes is TNFα associated Type 2 diabetes.

45. (canceled)

46. The method of claim 36 where in wherein the Type 2 diabetes is TNFα associated Type 2 diabetes.

47. The method of claim 37 where in wherein the Type 2 diabetes is TNFα associated Type 2 diabetes.
